(19) United States
(12) Patent Application Publication
(10) Pub. No.: US 2018/0289309 A1
(43) Pub. Date: Oct. 11, 2018

(54) INTELLIGENT STOP SHAKING DEVICE, SYSTEM AND METHOD

(71) Applicants: Robert CV Chen, Cupertino, CA (US); Tiffany YW Chen, Cupertino, CA (US)

(72) Inventors: Robert CV Chen, Cupertino, CA (US); Tiffany YW Chen, Cupertino, CA (US)

(21) Appl. No.: 16/006,830

(22) Filed: Jun. 12, 2018

Related U.S. Application Data
(63) Continuation-in-part of application No. 15/466,552, filed on Mar. 22, 2017, now abandoned.

(51) Int. Cl. A61B 5/00; A61B 5/06; A61B 5/11

(52) U.S. Cl. CPC A61B 5/4082; A61B 5/6822; A61B 5/067; A61B 2562/0219; A61B 5/11; A61B 5/6828; A61B 5/486; A61B 5/6825

(57) ABSTRACT

A system for inhibiting shaking includes a plurality of parallel tracks each to join a plurality of mini gyroscopes in series, the parallel tracks joined at one or more control junctures and disposed on a flexible substrate wrap. The system also includes a plurality of mini gyroscopes spaced apart in series by each of the plurality of parallel tracks, the mini gyroscopes to spin about an axis relative to a single parallel track at a variable angular momentum. The system additionally includes a plurality of accelerometers to differentiate shaking and purposeful movement of portions of the flexible substrate wrap and provide output thereof and feedback on shaking ablation. The system further includes a controller to control the variable angular momentum and the spin axis of the mini gyroscopes to ablate shaking relative to purposeful movement of portions of the substrate wrap based on accelerometer output.

### CROSS-REFERENCE TO RELATED APPLICATIONS

This application claims the benefit of the priority date of earlier filed U.S. Non-Provisional Utility application Ser. No. 15/446,552 filed Mar. 22, 2017 for Robert CV Chen and Tiffany YW Chen incorporated herein by reference in its entirety.

### BACKGROUND OF THE INVENTION

Parkinson's disease is a medical disorder whose primary symptom is excessive muscle contraction manifest by shaking. It is characterized by muscle rigidity, a slowing of physical movements, and in many cases tremor. At its worst, it can affect every muscle system in the body.

Parkinson's disease is a progressive, neurodegenerative disorder that affects movement, muscle control, and balance as well as numerous other functions. It is part of a group of conditions known as motor systems disorders. Parkinson's disease was named for James Parkinson, a general practitioner in London during the 19th century who first described the symptoms of the disease.

Symptoms describing Parkinson's disease are mentioned in the writings of medicine in India dating back to 5,000 BCE as well as in Chinese writings dating back approximately 2500 years. Parkinson's disease is the most common movement disorder and the second most common neurodegenerative disorder, the most common being Alzheimer's disease.

Parkinson's disease has an insidious onset, meaning it is slow to progress. The common early stage symptom is a tremor and an awkward movement: maximal when the limb is at rest and disappearing with voluntary movement and sleep. It affects to a greater extent the most distal part of the limb and at onset it typically appears in only a single arm or leg, becoming bilateral later. The major symptoms are a rest tremor, bradykinesia, muscle rigidity and posture gait disorder.

Adult-Onset Parkinson's Disease—This is the most common type of Parkinson's disease. The Parkinson's disease can significantly impair quality of life not only for the patients but for their families as well, and especially for the primary caregivers.

According to the American Parkinson's Disease Association, there are approximately 1.5 million people in the U.S. who suffer from Parkinson's disease—approximately 1-2% of people over the age of 60 and 3-5% of the population over age 85. The incidence of PD ranges from 8.6-19 per 100,000 people. Approximately 50,000 new cases are diagnosed in the U.S. annually. There are more than 2.0 million people in China who suffer from Parkinson's disease.

There has therefore been a long unsatisfied demand in the market place for a device, system and method of inhibiting shaking for Parkinson's victims.

### SUMMARY OF THE INVENTION

A disclosed device and system for inhibiting shaking comprises a plurality of parallel tracks comprising semi rigid connective material similar to cartilage on a flexible substrate wrap, the parallel tracks configured to provide a semi rigid route and inter placement of a plurality of devices received therein. The parallel tracks each configured to join mini gyroscope devices accumulated in series at any point on each of the parallel tracks, the mini gyroscopes configured to be routed along a single parallel track and be accumulated by an accumulating movement of a hand, leg or neck. The tracks are joined at one or more control junctures and disposed on a flexible substrate wrap. The mini gyroscopes are configured to spin about an axis relative to a single parallel track at a variable angular momentum. The system additionally includes a plurality of accelerometers inter placed between the mini gyroscopes in the parallel tracks configured to differentiate shaking and purposeful movement of portions of the flexible substrate wrap and provide output thereof and feedback on shaking ablation. The system further includes a controller configured to control the variable angular momentum and the spin axis of the mini gyroscopes to ablate shaking relative to purposeful movement of portions of the substrate wrap based on accelerometer output.

A disclosed method for an inhibition of shaking, the method comprises providing a plurality of parallel tracks each configured to join a plurality of mini gyroscopes in series, the parallel tracks joining one or more control junctures disposed on a flexible substrate wrap. The method also comprises providing a plurality of mini gyroscopes spaced apart in series by each of the plurality of parallel tracks, the mini gyroscopes spinning about an axis relative to a single parallel track at a variable angular momentum. Additionally, the method includes providing a plurality of accelerometers for differentiating shaking and purposeful moving of portions of the flexible substrate wrap and providing output thereof and feedback on shaking ablation. The method further includes providing a controller controlling the variable angular momentum and the spin axis of the mini gyroscopes to ablate shaking relative to purposeful movement of portions of the substrate wrap based on accelerometer output.

Other aspects and advantages of embodiments of the disclosure will become apparent from the following detailed description, taken in conjunction with the accompanying drawings, illustrated by way of example of the principles of the disclosure.

### BRIEF DESCRIPTION OF THE DRAWINGS

FIG. 1 is a back perspective view of the intelligent stop shaking necklace device and system in accordance with an embodiment of the present disclosure.

FIG. 1A is a cross sectional view of A-A shown in FIG. 1 in accordance with an embodiment of the present disclosure.

FIG. 1B is a cross sectional view of A-A shown in FIG. 1 in accordance with an embodiment of the present disclosure.

FIG. 1C is a cross sectional view of A-A shown in FIG. 1 in accordance with an embodiment of the present disclosure.

FIG. 2 is a front perspective view of the intelligent stop shaking necklace device and system in accordance with an embodiment of the present disclosure.

FIG. 3 is a closeup front perspective view of the intelligent stop shaking necklace device and system in accordance with an embodiment of the present disclosure.

FIG. 4 is a top anterior perspective view of the intelligent stop shaking hand glove device and system in accordance with an embodiment of the present disclosure.

FIG. 5 is a top posterior perspective view of the intelligent stop shaking hand glove device and system in accordance with an embodiment of the present disclosure.

FIG. 6 is a side perspective view of the intelligent stop shaking anklet device and system in accordance with an embodiment of the present disclosure.

FIG. 7 is a block diagram representation of the intelligent shake inhibiting device and system in accordance with an embodiment of the present disclosure.

FIG. 8 is a flow chart of a method for intelligently inhibiting shaking in accordance with an embodiment of the present disclosure.

FIG. 9 is a flow chart of implementation methods for intelligently inhibiting shaking in accordance with an embodiment of the present disclosure.

FIG. 10 is a depiction of a cell phone wireless telemetry and communication between the intelligent stop shaking device, system and computer program in accordance with an embodiment of the present disclosure.

Throughout the description, same reference numbers may be used to identify same or similar elements depicted in multiple embodiments. Although specific embodiments of the invention have been described and illustrated, the invention is not to be limited to the specific forms or arrangements of parts so described and illustrated. The scope of the invention is to be defined by the claims appended hereto and their equivalents.

### DETAILED DESCRIPTION

Reference will now be made to exemplary embodiments illustrated in the drawings and specific language will be used herein to describe the same. It will nevertheless be understood that no limitation of the scope of the disclosure is thereby intended. Alterations and further modifications of the inventive features illustrated herein and additional applications of the principles of the inventions as illustrated herein, which would occur to one skilled in the relevant art and having possession of this disclosure, are to be considered within the scope of the invention.

Throughout the present disclosure, the term ‘ablate’ refers to inhibiting, attenuating, lessening or countering shaking by mechanical means. The ablation of shaking may not remove the shaking but allow a user of the disclosure to have more purposeful use of their fingers, neck, legs, ankles, etc. The term ‘track’ refers to a connective physical structure for providing a common rigid route and inter placement of the mini gyroscopes and accelerometers onto the substrate glove, bracelet/choker or anklet. The track therefore accumulates and channels the angular momentum for the fingers and thumb of a user's hand. The term ‘shaking’ referred to in the present disclosure is synonymous with trembling and spasmodic movement associated with neurological seizures. The term ‘parallel’ refers to coincident multiple tracks parallel at some point but not necessarily parallel at all points between the multiple tracks. The term ‘accumulate’ refers to gather together in each track an increasing number of devices in the tracks at any point via a purposeful shaking of the device. Cartilage refers to a firm, touch, elastic and fibrous flexible connective material. The term ‘parallel’ used herein refers to collinear and coincident tracks which are substantially parallel at some points of distribution there between.

Dimensions detailed herein and in the drawings are intended to be a guide to nominal manufacturing dimensions. The detailed dimensions may vary by plus or minus ten percent taking into account manufacturing restraints and materials for various embodiments as recited, taught and suggested herein. The dimensions therefore are applicable to at least one embodiment but are not meant to be limiting to other embodiments of the disclosure.

Each iStopShaking® Device is powered by 25 to 30 mini gyroscopes (spinning at up to 40,000 rpm) per gyroscope. The gyroscopes are driven by long life battery. The mini gyroscopes are driven by a brushless DC (BLDC) driver controller. The BLDC drives one or more brushless slot less DC motors. The speed of the brushless slot less DC motor is up to 70,000 rpm and the torque is up to 48 mNm.

The controller board user interface is touch sensing buttons, I/O, oscillator, timer, motion detection sensor. The iStopShaking device works in conjunction with the gyroscope and the Accelerometer. The accelerometer is designed to measure non-gravitational acceleration. When the iStopShaking device is integrated with the accelerometer, it goes from a standstill to any velocity. The Accelerometer is designed to respond to the vibrations associated with such movement. It uses microscopic crystals that go under stress when vibrations occur, and from that stress a voltage is generated to create a reading on any acceleration. Accelerometers are important components to devices that track fitness and other measurements in the quantified self-movement.

Components of the disclosed iStopShaking tm devices are, On-off switch, High-Low intensity switch, sleep mode, Wireless CPU chip & Bluetooth connects with iStopShaking device with iPhone APP, iStopShaking® will also monitor eight activities and send data to patient's LCD screen on wrist and thru wireless connectivity thru wireless chip and Bluetooth to user's iPhone.

The iStopShaking devices also measure a broad range of vital signs, store/transmit data and sync with smartphones, tablets and PCs. iStopShaking devices has Built in sensors that will monitor and Measure heart rate, Blood oxygen sensor, Skin temperature sensor, Sleep sensor, Calories expended sensor,

Exercise, steps, walk distance sensor, and Exposure to radiation.

Each iStopShaking® device contains between 24 to 30 mini gyroscopes and accelerometers connected to “multiple tracks constructed inside hollow tube(s)”. The gyroscopes are connected in tracks built inside hollow tubes. Five hollow tubes with up to 30 gyroscopes are connected to brushless controller and brushless slot less DC compact motors. The brushless slot less DC compact motors have high acceleration, low noise and are connected to a power management (battery power source).

A gyroscope is a device that uses Earth's gravity to help determine orientation. Its design consists of a freely-rotating disk called a rotor, mounted onto a spinning axis in the center of a larger and more stable wheel.

The accelerometers measure acceleration i.e. how fast the gyroscope are speeding up or slowing down. Triaxial Accelerometers are used to sense both static (e.g. gravity) and dynamic (e.g. sudden starts/stops) acceleration along three cartesian axis. The accelerometer is used for tilt-sensing. An accelerometer is also used to sense gyroscope motion. Accelerometers will have a digital interface connected to wireless CPU with Bluetooth. The quantified-self movement build into the iStopShaking® device allow individuals to track all aspects of their daily lives, including their total activity, number of steps, food they eat, amount of sleep, heart rate, and mood.

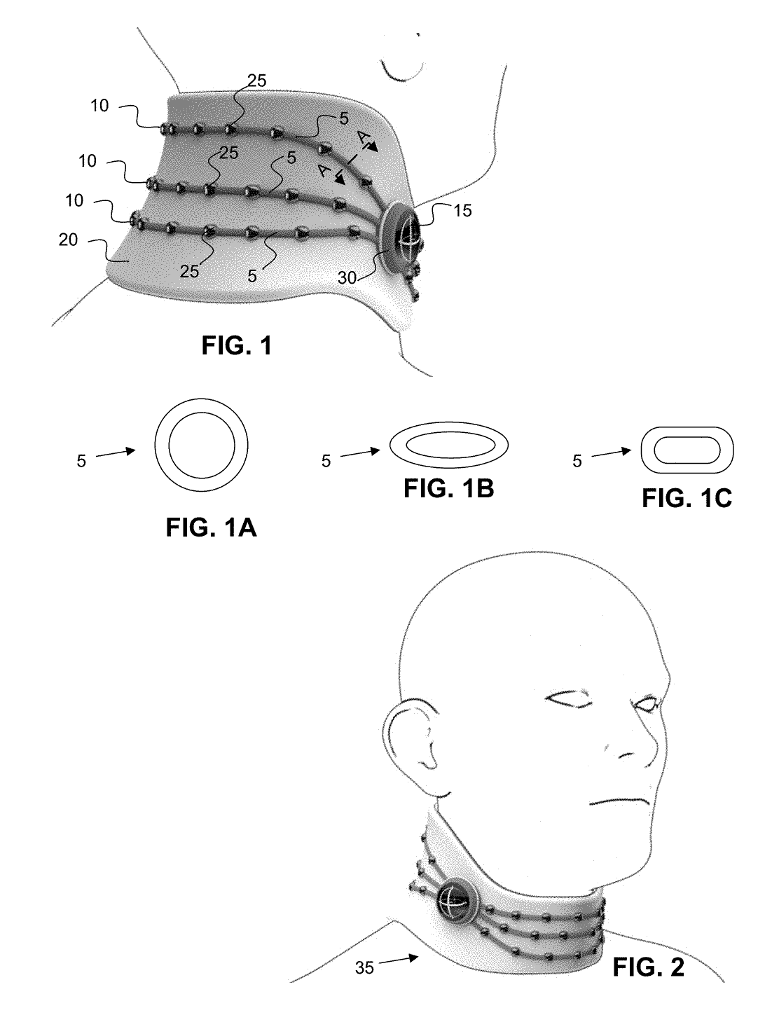

FIG. 1 is a back perspective view of the intelligent stop shaking necklace device and system in accordance with an embodiment of the present disclosure. The disclosed shaking inhibiting device comprises a plurality of mini gyroscopes 10 spaced apart in series by each of a plurality of parallel tracks 5, the mini gyroscopes 10 configured to spin about an axis relative to a single parallel track 5 at a variable angular momentum. The device also includes a plurality of accelerometers 25 configured to differentiate shaking and purposeful movement of portions of the flexible substrate wrap 20 and provide output thereof and feedback on shaking ablation. The device further includes a controller 30 configured to control the variable angular momentum and the spin axis of the mini gyroscopes to ablate shaking relative to purposeful movement of portions of the substrate wrap 20 based on the accelerometer output.

The mini gyroscopes 10 and the accelerometers 25 are depicted similarly from an outside appearance because placement and respective quantity may be determined by medical or technical personnel depending on the degree of sensitivity and control needed for the user and patient. In other words, more distributed accelerometer placement enables better determination of the purposeful and involuntary movement a patient suffers and therefore also allows better management and control of shaking ablation.

The parallel tracks 5 may be comprised of a semi rigid connective material similar to cartilage and therefore channel the angular momentum of the no shake gyroscopes. The tracks may be circular as in a bracelet, a necklace or an anklet or the tracks may be phalangeal as in the back of each finger and thumb. The tracks may therefore originate at bidirectional control junctures as in the bracelet, necklace and anklet or may originate at a unidirectional control juncture as at a back of a wrist for a hand glove application. The tracks may also therefore terminate at bidirectional control junctures or at the ends of fingers and a thumb respectively.

FIG. 1A is a cross sectional view of A-A shown in FIG. 1 in accordance with an embodiment of the present disclosure. A track 5 may be hollow and circular as depicted allowing the mini gyroscopes 10 and accelerometers 25 to ride inside the track according to accumulation movements of the flexible substrate wrap 20. A track 5 may also be solid and circular and the mini gyroscopes 10 and accelerometers 25 may ride the track on an outside thereof.

FIG. 1B is a cross sectional view of A-A shown in FIG. 1 in accordance with an embodiment of the present disclosure. A track 5 may be hollow and oval as depicted allowing the mini gyroscopes 10 and accelerometers 25 to ride inside the track according to accumulation movements of the flexible substrate wrap 20. A track 5 may also be solid and oval and the mini gyroscopes 10 and accelerometers 25 may ride the track on an outside thereof.

FIG. 1C is a cross sectional view of A-A shown in FIG. 1 in accordance with an embodiment of the present disclosure. A track 5 may be hollow and angular as depicted allowing the mini gyroscopes 10 and accelerometers 25 to ride inside the track according to accumulation movements of the flexible substrate wrap 20. A track 5 may also be solid and angular and the mini gyroscopes 10 and accelerometers 25 may ride the track on an outside thereof.

Accumulation movements include purposeful flicking movements of a hand or a leg to shake the mini gyroscopes and the accelerometers to the tips of a user's fingers for example. The mini gyroscopes may also be brought back nearer to a wrist of the user via gravity or taking the flexible substrate wrap off the user and flicking it in a manner to redistribute the mini gyroscopes and accelerometers from the accumulated positions. The accumulation movements are differentiated by other purposeful movements of the user's hand as if the user were trying to flick water from his or her finger tips. Other purposeful movements include writing, gentle washing, squeezing, pointing, etc from most everyday conscious movements. Shaking movements are not conscious movements and are involuntary as a result of disease or deterioration of the nerves and muscles of the body. Bodily functions include heart rate, diastolic and systolic pressures, oxygen content, and relative hormonal components in the blood of a user. Any point of accumulation refers to distances from a controller juncture along a track.

FIG. 2 is a front perspective view of the intelligent stop shaking necklace device and system in accordance with an embodiment of the present disclosure. Reference numbers and similar claimed limitations are depicted in FIG. 2 as in FIG. 1 for the intelligent stop shaking necklace 35. The front view depicted includes a similar configuration of respective elements. An embodiment of the shaking inhibiting system further comprises a circuit in the controller configured to set an accelerometer acceleration and a spin axis of the mini gyroscopes equal to a an acceleration and a direction of the purposeful movement determined by the accelerometer output during a purposeful movement. The disclosed system further comprises a circuit for differentiation of accelerometer acceleration output from shaking greater than an accelerometer acceleration from purposeful movement.

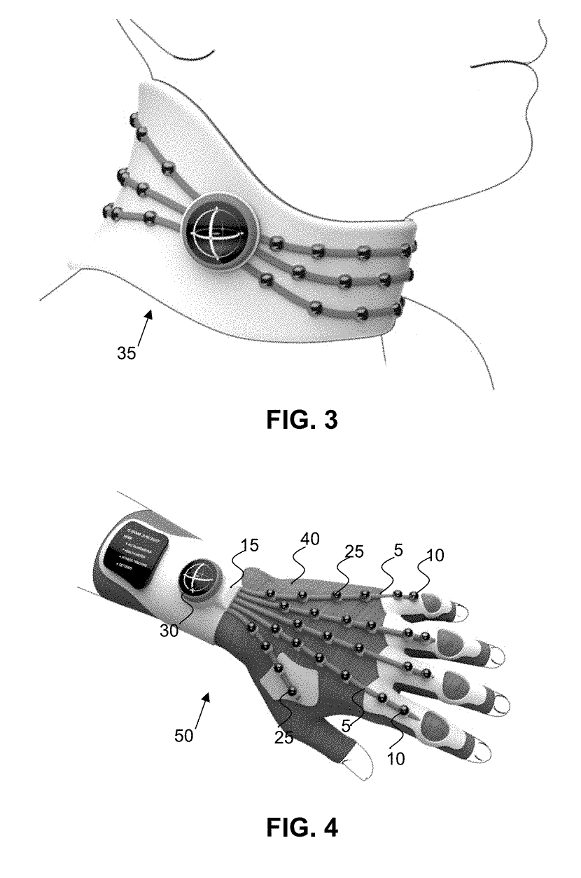

FIG. 3 is a closeup front perspective view of the intelligent stop shaking necklace device and system in accordance with an embodiment of the present disclosure. Reference numbers and similar claimed limitations are depicted in FIG. 3 as in FIG. 2 for the intelligent stop shaking necklace 35. The front view depicted includes a similar configuration of respective elements. Also, a circuit is included for incorporating feedback on shaking ablation provided by the accelerometers into an increase or a decrease of an intensity of the angular momentum and the spin axis of the mini gyroscopes. A comprised flexible wrap is configured as a substrate for the plurality of mini gyroscopes and the plurality of parallel tracks and one or more control junctures, the substrate and portions thereof comprising a hand glove, a bracelet and an anklet.

FIG. 4 is a top anterior perspective view of the intelligent stop shaking hand glove device and system in accordance with an embodiment of the present disclosure. The disclosed shaking inhibiting hand glove device 50 comprises a plurality of mini gyroscopes 10 spaced apart in series by each of a plurality of parallel tracks 5, the mini gyroscopes 10 configured to spin about an axis relative to a single parallel track 5 at a variable angular momentum. The device also includes a plurality of accelerometers 25 configured to differentiate shaking and purposeful movement of portions of the flexible substrate wrap 40 and provide output thereof and feedback on shaking ablation. The device further includes a controller 30 configured to control the variable angular momentum and the spin axis of the mini gyroscopes to ablate shaking relative to purposeful movement of portions of the substrate wrap 40 based on the accelerometer output.

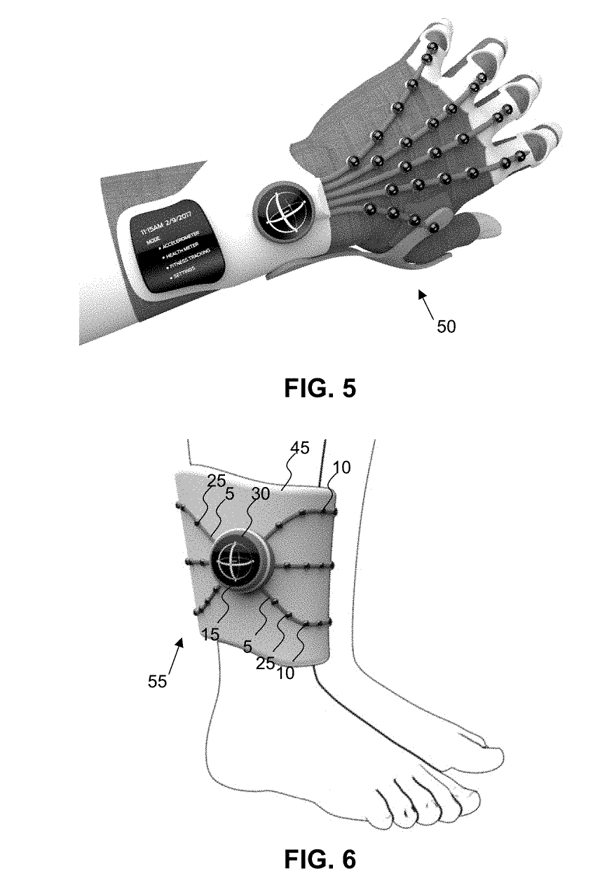

FIG. 5 is a top posterior perspective view of the intelligent stop shaking hand glove device and system in accordance with an embodiment of the present disclosure. Reference numbers and similar claimed limitations are depicted in FIG. 5 as in FIG. 4 for the intelligent stop shaking hand glove 50. The flexible substrate wrap comprises a hand glove 40 with finger and thumb portions and finger and thumb parallel tracks comprising a control juncture adjacent a wrist of the glove. In an embodiment, at least one controller portion is disposed at one control juncture thereof and another controller portion is disposed at another juncture thereof. An ablation intensity switch is configured to allow a user of the device to determine a mini gyroscope angular momentum from one of a plurality of ablation intensity gradations.

FIG. 6 is a side perspective view of the intelligent stop shaking anklet device and system in accordance with an embodiment of the present disclosure. The flexible substrate wrap also comprises an anklet with three parallel tracks configured to circumnavigate an ankle. The flexible substrate wrap may additionally comprises a bracelet with three parallel tracks circumnavigating a neck. The disclosed shaking inhibiting anklet device 55 comprises a plurality of mini gyroscopes 10 spaced apart in series by each of a plurality of parallel tracks 5, the mini gyroscopes 10 configured to spin about an axis relative to a single parallel track 5 at a variable angular momentum. The device also includes a plurality of accelerometers 25 configured to differentiate shaking and purposeful movement of portions of the flexible substrate wrap 45 and provide output thereof and feedback on shaking ablation. The device further includes a controller 30 configured to control the variable angular momentum and the spin axis of the mini gyroscopes to ablate shaking relative to purposeful movement of portions of the substrate wrap 45 based on the accelerometer output.

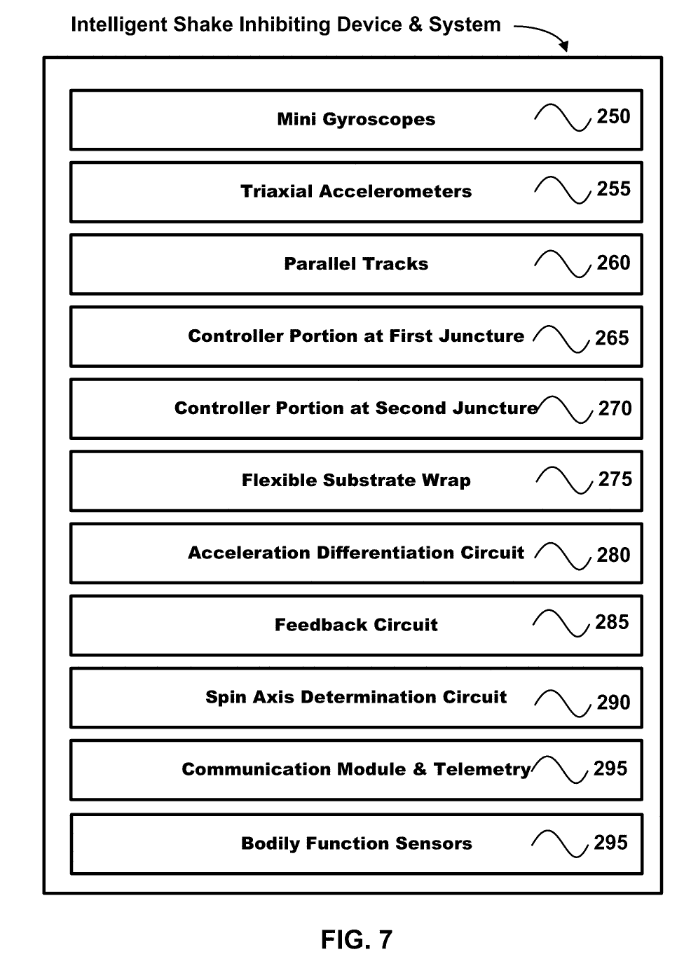

FIG. 7 is a block diagram representation of the intelligent shake inhibiting device and system in accordance with an embodiment of the present disclosure. The system includes mini gyroscopes 250, mini accelerometers 255, parallel tracks 260, a controller portion at a first juncture 265, a controller portion at a second juncture 270, a flexible substrate wrap 275, an acceleration differentiation circuit 280, a feedback circuit 285, a spin axis determination circuit 290, a communication module & telemetry 295 and bodily function sensors 295 as disclosed herein.

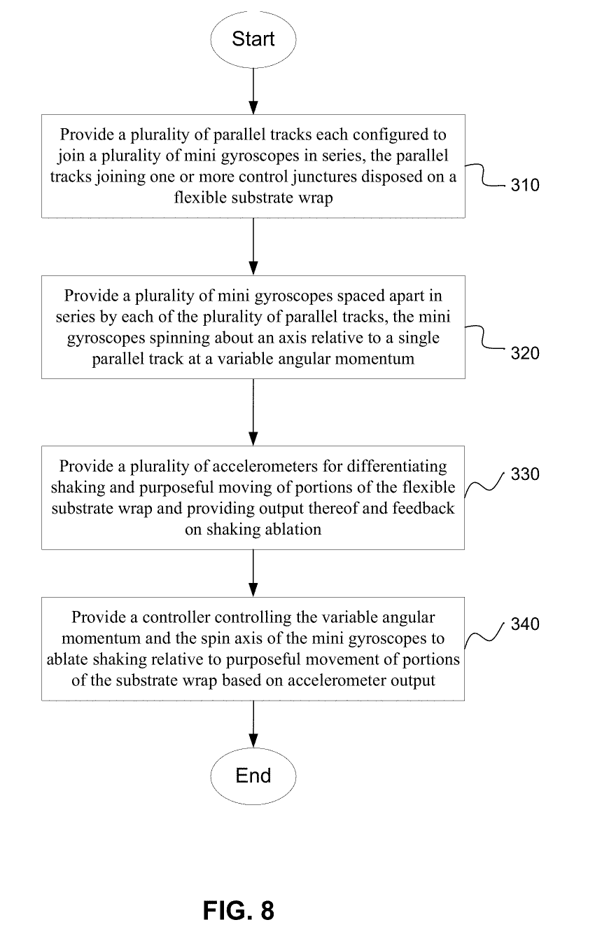

FIG. 8 is a flow chart of a method for intelligently inhibiting shaking in accordance with an embodiment of the present disclosure. The method includes providing 310 a plurality of parallel tracks each configured to join a plurality of mini gyroscopes in series, the parallel tracks joining one or more control junctures disposed on a flexible substrate wrap. The method also comprises providing 320 a plurality of mini gyroscopes spaced apart in series by each of the plurality of parallel tracks, the mini gyroscopes spinning about an axis relative to a single parallel track at a variable angular momentum. Additionally, the method includes providing 330 a plurality of accelerometers for differentiating shaking and purposeful moving of portions of the flexible substrate wrap and providing output thereof and feedback on shaking ablation. The method further includes providing 340 a controller controlling the variable angular momentum and the spin axis of the mini gyroscopes to ablate shaking relative to purposeful movement of portions of the substrate wrap based on accelerometer output.

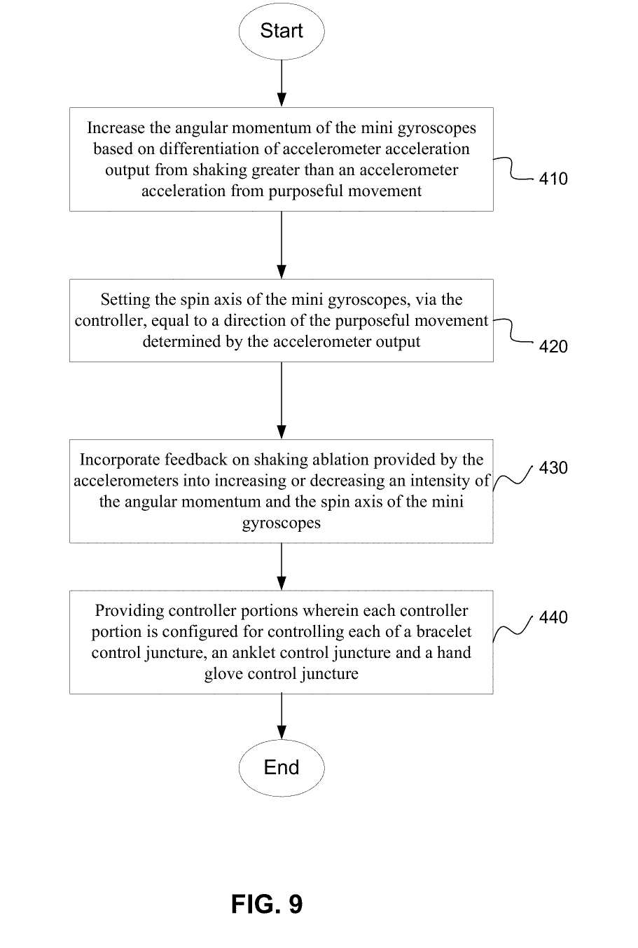

FIG. 9 is a flow chart of implementation methods for intelligently inhibiting shaking in accordance with an embodiment of the present disclosure. The implementation method includes increasing 410 the angular momentum of the mini gyroscopes based on differentiation of accelerometer acceleration output from shaking greater than an accelerometer acceleration from purposeful movement. The implementation method also includes setting 420 the spin axis of the min gyroscopes via the controller equal to a direction of the purposeful movement determined by the accelerometer output. The method additionally includes incorporating feedback 430 on shaking ablation provided by the accelerometers into increasing or decreasing an intensity of the angular momentum and the spin axis of the mini gyroscopes. The method further includes providing 440 controller portions wherein each controller portion is configured for controlling each of a bracelet control juncture, an anklet control juncture and a hand glove control juncture.

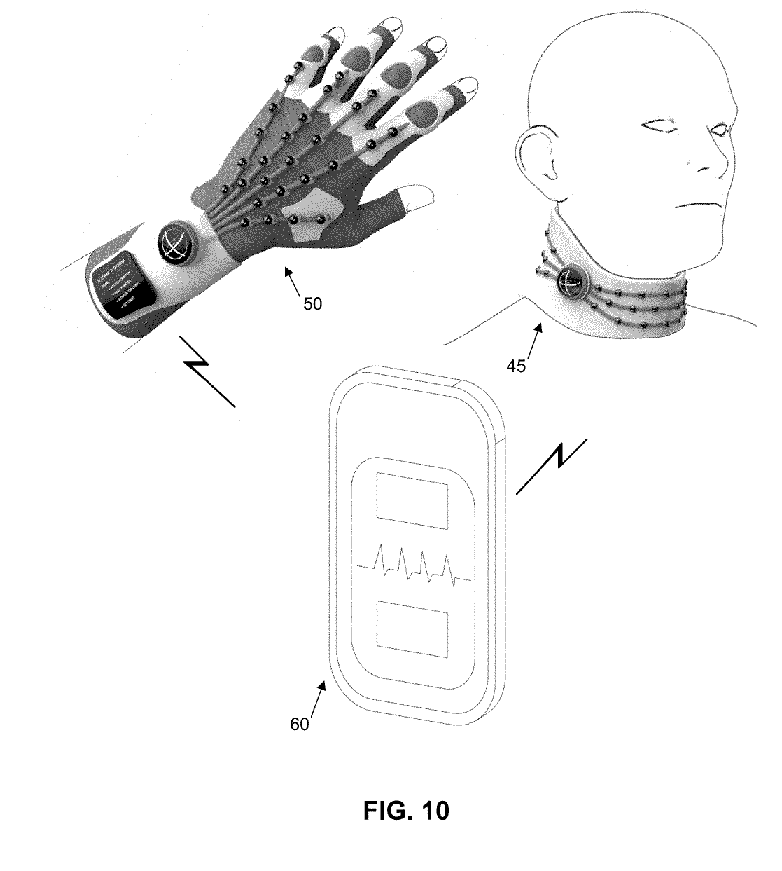

FIG. 10 is a depiction of a cell phone wireless telemetry and communication between the intelligent stop shaking device, system and computer program in accordance with an embodiment of the present disclosure. The depiction includes the flexible substrate wrap 45, the hand glove 50 and a cell phone 60. The mini gyroscopes comprise miniaturized electro-mechanical gyroscopes and accelerometer elements made using microfabrication techniques (MEMS). Also an electronic touch display is in communication with the controller, the display configured for an input and output of processed bodily functions and device status. A communications module and protocol thereof are included for wireless telemetry and communication with a cell phone. Additionally, sensors are included which are configured to sense, monitor, record and communicate bodily functions.

The intelligent shaking inhibiting devices, system and method will send data to a cell phone or another remote electronic device including time domain and relevance to trembling and shaking frequency, degree of trembling and shaking. This telemetry capability allows a user and patient of the disclosure to track episodes and use graphics and memory functions to manage their health care and ablation of their trembling and shaking. Applications are also included in embodiments which allow the user and patient to program response to tremors and shaking episodes via predetermined inhibiting intensity to allow health care management during sleep.

The present disclosure therefore meets the long felt need in the market for a device, system, computer program and method for the management and ablation of involuntary shaking, trembling and spasmodic events including seizures. The present disclosure also provides electronic means for the management of many such intelligent no shake devices on all appendages of a patient through a central or distributed application and wireless management including personal digital devices and the internet cloud.

Although the operations of the method(s) herein are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

While the forgoing examples are illustrative of the principles of the present disclosure in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the disclosure be limited, except as by the specification and claims set forth herein.

What is claimed is:

1. A shaking inhibiting device, comprising:
a plurality of parallel tracks comprising semi rigid connective material similar to cartilage disposed on a flexible substrate wrap, the parallel tracks configured to provide a semi rigid route for and an inter placement of a plurality of devices;
a plurality of mini gyroscope devices accumulated in series at any point on each of the plurality of parallel tracks, the mini gyroscopes configured to be routed along a single parallel track and spin at a variable angular momentum;
a plurality of accelerometer devices inter placed between the mini gyroscopes in the parallel tracks, the accelerometers configured to differentiate shaking and purposeful movement of portions of the flexible substrate wrap and provide output thereof and feedback on shaking ablation; and
a controller configured to control the variable angular momentum and the spin axis of the mini gyroscopes to ablate shaking relative to purposeful movement of portions of the substrate wrap based on the accelerometer output.

2. The shaking inhibiting system of claim 1, wherein the parallel tracks are hollow and allow the mini gyroscopes and the accelerometers to travel therein according to an accumulation movement of a user.

3. The shaking inhibiting device of claim 1, further comprising a circuit for differentiation of accelerometer acceleration output from shaking greater than an accelerometer acceleration from purposeful movement.

4. The shaking inhibiting device of claim 1, further comprising a circuit for incorporating feedback on shaking ablation provided by the accelerometers into an increase or a decrease of an intensity of the angular momentum and the spin axis of the mini gyroscopes.

5. The shaking inhibiting device of claim 1, further providing a flexible wrap configured as a substrate for the plurality of mini gyroscopes and the plurality of parallel tracks and one or more control junctures, the substrate and portions thereof comprising a hand glove, a necklace and an anklet.

6. A shaking inhibiting system, comprising:
a plurality of parallel tracks each configured to join a plurality of mini gyroscopes in series, the parallel tracks joined at one or more control junctures and disposed on a flexible substrate wrap;
a plurality of mini gyroscopes spaced apart in series by each of the plurality of parallel tracks, the mini gyroscopes configured to spin about an axis relative to a single parallel track at a variable angular momentum;
a plurality of accelerometers configured to differentiate shaking and purposeful movement of portions of the flexible substrate wrap and provide output thereof and feedback on shaking ablation; and
a controller configured to control the variable angular momentum and the spin axis of the mini gyroscopes to ablate shaking relative to purposeful movement of portions of the substrate wrap based on accelerometer output.

7. The shaking inhibiting system of claim 6, wherein the flexible substrate wrap comprises a hand glove with finger and thumb portions and finger and thumb parallel tracks comprising a control juncture adjacent a wrist of the glove.

8. The shaking inhibiting system of claim 6, wherein the flexible substrate wrap comprises an anklet with three parallel tracks configured to circumnavigate an ankle.

9. The shaking inhibiting system of claim 6, wherein the flexible substrate wrap comprises a bracelet with three parallel tracks circumnavigating a neck.

10. The shaking inhibiting system of claim 6, wherein at least one controller portion is disposed at one control juncture thereof and another controller portion is disposed at another juncture thereof.

11. The shaking inhibiting device of claim 6, further comprising an ablation intensity switch configured to allow a user of the device to determine a mini gyroscope angular momentum from one of a plurality of ablation intensity gradations.

12. The shaking inhibiting system of claim 6, wherein the parallel tracks are solid and allow the mini gyroscopes and the accelerometers to travel thereon according to an accumulation movement of a user.

13. The shaking inhibiting system of claim 6, further comprising an electronic touch display in communication with the controller, the display configured for an input and output of processed bodily functions and device status.

14. The shaking inhibiting system of claim 6, further comprising a communications module and protocol thereof for wireless telemetry and communication with a cell phone.

15. The shaking inhibiting system of claim 6, further comprising sensors configured to sense, monitor, record and communicate bodily functions.

16. A method for inhibiting shaking, the method comprising:
providing a plurality of parallel tracks comprising semi rigid connective material similar to cartilage on a flexible substrate wrap, each track configured to join a plurality of mini gyroscopes in series, the parallel tracks joining one or more control junctures disposed on a flexible substrate wrap;
providing a plurality of mini gyroscopes spaced apart in series by each of the plurality of parallel tracks, the mini gyroscopes spinning about an axis relative to a single parallel track at a variable angular momentum;
providing a plurality of accelerometers for differentiating shaking and purposeful moving of portions of the flexible substrate wrap and providing output thereof and feedback on shaking ablation; and
providing a controller controlling the variable angular momentum and the spin axis of the mini gyroscopes to ablate shaking relative to purposeful movement of portions of the substrate wrap based on accelerometer output

17. The method for inhibiting shaking of claim 16, further comprising increasing the angular momentum of the mini gyroscopes based on differentiation of accelerometer acceleration output from shaking greater than an accelerometer acceleration from purposeful movement.

18. The method for inhibiting shaking of claim 16, further comprising the controller setting the spin axis of the mini gyroscopes equal to a direction of the purposeful movement determined by the accelerometer output.

19. The method for inhibiting shaking of claim 16, further comprising incorporating feedback on shaking ablation provided by the accelerometers into increasing or decreasing an intensity of the angular momentum and the spin axis of the mini gyroscopes.

20. The method for inhibiting shaking of claim 16, further comprising a plurality of controller portions wherein each controller portion is configured for controlling each of a necklace control juncture, an anklet control juncture and a hand glove control juncture.
